(19) United States
(12) Patent Application Publication
(10) Pub. No.: US 2003/0077632 A1
(43) Pub. Date: Apr. 24, 2003

(54) Nuclear bodies

(76) Inventors: Jeanne B. Lawrence, Mapleville, RI (US); Kelly P. Smith, Shrewsbury, MA (US); Meg Byron, Sterling, MA (US)

Correspondence Address: FISH & RICHARDSON PC, 225 FRANKLIN ST, BOSTON, MA, 02110, US

(21) Appl. No.: 10/217,495

(22) Filed: Aug. 12, 2002

Related U.S. Application Data
(60) Provisional application No. 60/311,456, filed on Aug. 10, 2001.

(51) Int. Cl. C12Q 1/68; G01N 33/567

(52) U.S. Cl. 435/6; 435/7.21

(57) ABSTRACT

The compositions and methods described herein are based, at least in part, on fundamental observations concerning nuclear organization and gene expression. More specifically, the studies described below have revealed a new type of nuclear body or intra-nuclear organelle, which is referred to herein as a PAN body (an acronym for PML body adjacent nuclear body). The evidence collected to date supports a role for PAN bodies in nuclear function, gene regulation (or expression), and disease. For example, the studies reveal a structural relationship between PAN bodies and PML bodies, which are implicated in cell proliferation and apoptosis. This relationship and others are discussed further below. Accordingly, the invention features isolated or purified PAN bodies and therapeutic (including prophylactic), diagnostic, and screening methods that utilize PAN bodies.

The present application claims the benefit of the filing date of U.S. Ser. No. 60/311,456, which was filed on Aug. 10, 2001. The contents of U.S. Ser. No. 60/311,456 are hereby incorporated by reference in the present application in their entirety.

### GOVERNMENT FUNDING

Some of the work described herein was carried out with funds from the United States Government (under grant numbers MCB-0212963, M53234, and GM49254 awarded by the National Institutes of Health). Therefore, the Government has certain rights in the invention.

### TECHNICAL FIELD

This invention relates to structures within the nuclei of biological cells and methods in which these structures can be identified, isolated, and used (to, for example, identify various cells or substances).

### BACKGROUND

The nuclei within biological cells contain a number of distinct, non-membrane bound compartments such as SC35 domains, Cajal bodies, and PML (promyelocytic leukemia) bodies, in which different sets of macromolecules concentrate (Lamond and Earnshaw, Science 280:547-533, 1998; Shopland and Lawrence, J. Cell Biol. 150:F1-4, 2000). Studies to date indicate that SC35 domains (the 10-30 prominent snRNP speckles) are enriched in factors involved in pre-mRNA production (Moen et al., Human Mol. Genet. 4:1779-1789, 1995), whereas Cajal bodies contain factors involved in metabolism of both pre-mRNAs and nucleolar RNAs (Gall, Ann. Rev. Cell. Dev. Biol. 16:273-300, 2000). PML bodies are also referred to as PODs (PML oncogenic domains; Dyck et al., Cell 76:333-343, 1994; Weis et al., Cell 76:345-356, 1994; see also U.S. Pat. No. 6,319,663), and they were originally identified as ND10 (Nuclear Dot 10; Ascoli, Mol. Endocrinol. 15:485-500, 1991). There is substantial interest in PML bodies because of their intriguing connection to cancer. PML bodies contain multiple factors involved in gene regulation, growth control, and apoptosis, including the p53 tumor suppressor protein, and the breakdown of PML bodies is a hallmark of leukemic cells.

### SUMMARY OF THE INVENTION

The compositions and methods described herein are based, at least in part, on fundamental observations concerning nuclear organization and gene expression. More specifically, the studies described below have revealed a new type of nuclear body or intra-nuclear organelle, which is referred to herein as a PAN body (an acronym for PML body adjacent nuclear body). The evidence collected to date supports a role for PAN bodies in nuclear function, gene regulation (or expression), and disease. For example, the studies reveal a structural relationship between PAN bodies and PML bodies, which are implicated in cell proliferation and apoptosis. This relationship and others are discussed further below.

Accordingly, the invention features isolated or purified PAN bodies and therapeutic (including prophylactic), diagnostic, and screening methods that utilize PAN bodies. Generally, each of the methods described herein assesses some aspect of PAN expression, such as the number, size, content, or location of PAN bodies. Thus, when assessing PAN expression, one may observe an appearance, disappearance, distortion, or dislocation of the PAN body per se (as a whole) or, alternatively, a change in the content of the PAN body (e.g., a pathogen may direct a protein (either a cellular protein (e.g., an oncoprotein, transcription factor, or some other agent that regulates gene expression) or one from the pathogen) to an already formed PAN body.

More specifically, the invention features methods of identifying and characterizing a PAN body within a cell. The methods can be carried out by, for example, contacting a cellular nucleus with two antibodies: a first antibody that specifically binds the nuclear body or a protein therein and a second antibody that specifically binds a PML body or a protein therein. If the first antibody binds a nuclear body adjacent to a PML body (recognized as such by binding with the second antibody), then the nuclear body is a PAN body. The first and second antibodies need not be applied in any particular order and they may be applied to the cell or tissue simultaneously. Preferably, each antibody will be labeled in a way that allows it to be detected and distinguished from the other antibody used (from example, the antibodies, or secondary antibodies that bind thereto, may be labeled with different fluorophores). The cells can be obtained from a patient (e.g., a human patient who is suspected of having an infection or a cancer (e.g., the cells can be obtained from a biopsy)) and they may be subjected to immunohistochemistry directly or following a period in cell culture. If only the expression of PAN bodies is assessed (vs., for example, their location within the nucleus), the method can be carried out on homogenized tissue (e.g., one can carry out a Western blot). While antibodies are particularly useful, any agent (e.g., a nucleic acid) that specifically binds a nuclear body (i.e. a PAN body) can be used. When an antibody is used, it can be an anti-FLAG antibody, although the invention is not so limited. Any antibody that specifically binds the PAN body, or a protein therein (be it cellular or viral (or that of another type of pathogen) is useful. Alternatively, or in addition, one can examine more than the relationship between PAN and PML bodies. In other embodiments, the PAN body is further characterized by determining whether it is adjacent to an SC35 domain and/or a Cajal body. These methods can be carried out in the same manner as those just described, but antibodies or other specific labels for SC35 and/or Cajal bodies will be used. The relationship between PAN, PML, SC35 and Cajal bodies is described further below.

The invention also features methods of inducing the formation of (or changing the content of) a PAN body in a cell (e.g., a cultured cell, which can be a human cell). The method can be carried out by, for example, exposing a cell to media conditioned by prior exposure to PAN-positive cells or by exposing the cell to a pathogen (e.g., a bacterium or mycobacterium, a virus, or fungus). In the event the PAN-inducing agent is a virus, it can be a virus associated with a cancer (e.g., a leukemia, a liver cancer, or a cancer of the reproductive tract (e.g., ovarian cancer)) or an active ingredient thereof (e.g., a viral protein). Induction can be detected or confirmed by treating the cells exposed to the conditioned medium or the pathogen to an antibody or other agent that specifically binds a PAN body, or one or more proteins therein. As is true for other methods described herein, any agent that allows one to specifically recognize a PAN body (or other nuclear structure) is useful; the methods of the invention are not limited to those that employ antibodies, nor to those that employ any particular cell type. Here to, visualizing PAN bodies by any type of microscopy, including electron microscopy, can be done.

The invention also features methods of determining whether a cell (e.g. a cell in culture or in vivo, such as a human cell), has (a) been exposed to a pathogen (here again, the pathogen can be any pathogen, including bacteria and viruses) (b) is malignant or (c) is at risk of becoming malignant. The method includes determining whether the cell expresses PAN bodies (which can be detected visually or by way of assays such as Northern or Western blots that detect PAN-specific nucleic acid or protein expression, respectively). The presence of PAN bodies, or the pathogen-induced inclusion of a PAN-specific protein (be it cellular of non-cellular) within the PAN bodies, indicates that the cell has been exposed to a pathogen, is malignant, or is at risk of becoming malignant. Where PAN-specific antibodies are used (e.g., an anti-FLAG antibody), the assay may be carried out by immunoassay or immunohistochemistry.

In another aspect, the invention features a method of screening a test compound (e.g., a protein, peptide, nucleic acid, or a biological or non-biological chemical entity), to identify a potential therapeutic agent (e.g., an anti-viral or chemotherapeutic agent). The method includes providing a cell having a visible PAN body or a visible plurality of PAN bodies, contacting the cell with the test compound, and evaluating the effect of the test compound on the PAN body or PAN bodies, wherein disappearance of the PAN body, a reduction in the number of PAN bodies, or a disassociation between one or more PAN bodies and one or more PML bodies indicates that the test compound is a potential therapeutic agent.

In another aspect, the invention features methods of determining whether the state of PAN bodies within a diseased cell (e.g., a cell that is infected with a virus or that is malignant), can serve as a marker for disease. The method includes providing an apparently healthy cell and a cell that is diseased, and determining whether and, optionally, where, PAN bodies are expressed within the healthy cell and within the diseased cell, wherein a difference in PAN body expression between the apparently healthy cell and the cell that is diseased indicates that the state of PAN bodies are a marker for disease. In this method, the state of the PAN bodies in the diseased cell before and after exposure to a therapeutic agent is compared to an untreated apparently healthy cell. A beneficial effect is derived from the therapeutic agent if the PAN bodies of the diseased cell are in a state reminiscent of the state of PAN bodies in the healthy untreated cell.

In another aspect, the invention features a method of isolating a PAN body. The method includes providing cell nuclei, disrupting the nuclei in a solution containing divalent ions, e.g., magnesium ion concentration of about 0.5-1.0 mM, passing the disrupted nuclei over a Percoll-sucrose gradient at a first pH, e.g., pH 7.0, 7.2, 7.4, 7.6, or 7.8 and subsequently passing the resulting eluate over a Percoll-sucrose gradient at a higher second pH, e.g., pH 7.8, 8.0, 8.1, 8.2, 8.3, 8.4, or 8.5, to obtain enriched fractions of PAN bodies, and evaluating the enriched fractions for the presence of PAN bodies. Fractions of disrupted nuclei can be passed over Percoll-Sucrose or Sucrose gradients many times, e.g., two, three, four, or five times.

In yet another aspect, the invention features a PAN body isolated by the method above. In another embodiment, the PAN body is found associated adjacent to a PML body and an SC35 body in a 1:1:1 stoichiometry when present in the nucleus of a biological cell, forming a triad. In another embodiment, the PAN body is associated adjacent to a PML body in about a 1:1 stoichiometry when present in the nucleus of a biological cell, forming a twin with the PML body.

Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, the drawings, and the claims.

### BRIEF DESCRIPTION OF THE DRAWINGS

FIG. 1 is an electron micrograph of a nuclear doublet.

FIGS. 2A-2C are photomicrographs. FIG. 2A focuses on the nucleus of a cell and shows the location of PML bodies by virtue of an anti-Daxx antibody bound to fluorochrome-conjugated secondary antibody; FIG. 2B focuses on the same nucleus and shows the location of PAN bodies by virtue of an anti-FLAG® antibody bound to fluorochrome-conjugated secondary antibody; and FIG. 2C focuses on the same nucleus and shows the location of PML bodies and PAN bodies adjacent to each (an overlay of FIGS. 2A and 2B).

FIG. 3 is a photomicrograph focused on the nucleus of a cell showing the location of PAN bodies and centromeres not associated with each other as seen by anti-FLAG® and anti-centromere autoimmune serum bound to fluorochrome-conjugated secondary antibody.

FIG. 4A is a photomicrograph focused on the nucleus of a cell grown in conditioned medium showing the location of PAN bodies as seen by anti-FLAG® antibody bound to fluorochrome-conjugated secondary antibody.

FIG. 4B is a photomicrograph focused on the nucleus of a cell grown in non-conditioned media showing the absence of PAN bodies.

FIG. 5A-5D are photomicrographs of the same cell showing staining with different combinations of antibodies. FIG. 5A is a staining with antibodies recognizing PML (anti-Daxx) and SC35 domains (anti-SRm300). FIG. 5B is a staining of PAN bodies (anti-FLAG) and PML bodies (anti-Daxx). FIG. 5C. is a staining of PAN bodies (anti-FLAG) and SC35 domains (anti-SRm300). FIG. 5D is an overlay showing a triple staining of PAN bodies (anti-FLAG), PML bodies (anti-Daxx), and SC35 domains (anti-SRm300).

FIG. 6 is a schematic of a procedure to isolate PAN bodies.

### DETAILED DESCRIPTION

The interior of cellular nuclei has long been perceived as relatively simple, despite the remarkable complexity of its functions. Recently, however, a growing number of non-membrane bound compartments (or “domains” or “bodies”) have been identified that are characterized by discrete accumulations of specific subsets of factors (reviewed in Huang and Spector, J. Cell Biochem 62:191-197, 1996; Matera, Trends in Cell Biol. 9:302-309,1999; and Moen et al., Hum. Mol. Genet. 4:1779-1789 1995). Most RNA metabolic factors concentrate in discrete compartments, and findings now show that a host of gene regulatory factors also localize in largely unexplored nuclear bodies.

PAN Bodies and their Association with PML Bodies

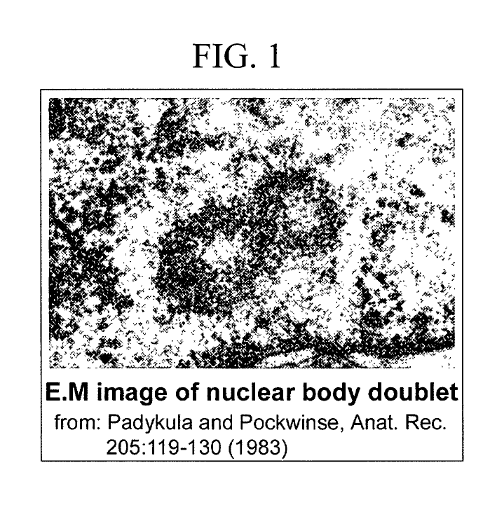

PAN bodies, as seen by a specific and reproducible cross reactivity with an anti-FLAG antibody (see the Examples, below), are bright, discrete nuclear bodies. We refer to PAN bodies as “bodies,” rather than “domains” or “foci,” due to their characteristically round shape (determined by 3-D reconstructions of serial images). PAN bodies have a substantially regular shape, unlike more irregularly shaped protein aggregates, which are typically referred to as foci. By immunofluorescence, PAN bodies were found adjacent to other nuclear structures, including PML bodies (i.e., PAN bodies are juxtaposed or spatially associated with PML bodies); they do not appear to be colocalized or “within” those bodies. The juxtaposition of these two bodies is reminiscent of an earlier observation made by electron microscopy in which nuclear bodies were seen in pairs (FIG. 1; Padykula and Pockwinse, Anat. Rec. 205:119-130, 1983; see also Ascoli and Maul, J. Cell Biol. 112:785-795, 1991), although the identity of those bodies was not ascertained (nor were they isolated).

The structures that partner with PAN bodies, PML bodies, are complex and interesting structures. They were first observed using an autoimmune serum (Ascoli and Maul, J. Cell Biol. 112:785-795, 1991) (as noted elsewhere, that serum and later developed antibodies can be used to identify PML bodies and characterize their location within a cell) and they were found to contain the PML protein, which is essential for PML nuclear body formation. The PML gene is fused to the retinoic acid receptor gene in the t(15; 17) translocation of acute promyelocytic leukemia (APL), resulting in the breakdown of PML domains (de The et al., Cell 66:675-84, 1991; Goddard et al., Science. 254:1371-4, 1991; Kakizuka et al., Cell 66:663-674, 1991; and Pandolfi et al., Oncogene 6:1285-1292, 1991). This breakdown is a hallmark of APL (Dyck et al., Cell 76:333-343, 1994; Weis et al., Cell 76:345-356, 1994). To date, over a dozen different proteins have been found in PML bodies. These proteins are involved in a number of different cellular processes including tumor suppression (pRB, PML, p53), growth control, apoptosis (p53), transcription regulation (p53, pRB, Daxx, Sp100, Sp140) and translation initiation (eIF-4, INT-6) (reviewed in Zhong et al., Nature Cell Biol. 2:E85-90, 2000), and their presence implicates PML bodies in these processes. On average, PML bodies are about 0.3-1.0 microns in diameter, and PAN bodies are similar in size and shape to PML bodies. Thus, the PAN bodies of the invention can be about 0.3, 0.4, 0.5, 0.6, 0.7, 0.8, 0.9, or 1.0 micron in diameter.

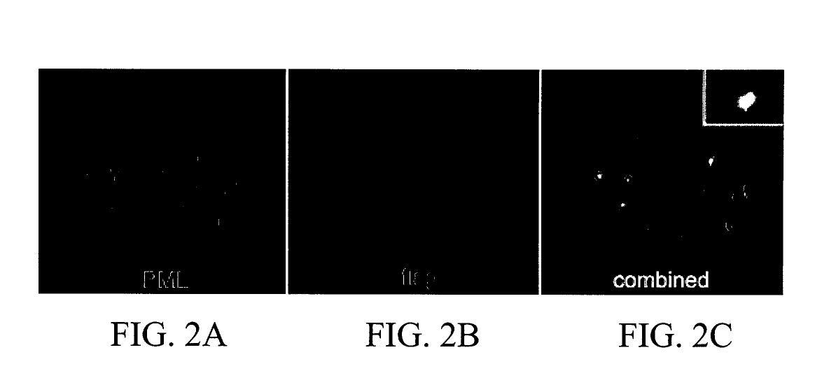

Consistent with their defined structure, PAN bodies show a consistent and fairly evenly spaced distribution in the nuclei with a reproducible, apparently round morphology that, as noted above, is similar to that of PML bodies. Further analysis showed about 80% of the PAN bodies within the nucleus were tightly coupled, but non-overlapping, with PML domains, and vice versa. FIGS. 1A-1C are photomicrographs of the nucleus of the same cell, illuminated to reveal PML bodies (FIG. 2A), PAN bodies (FIG. 2B), and both PML and PAN bodies (FIG. 2C; note the positioning adjacent to one other). While the PAN bodies are not always paired with PML bodies, their association is too frequent to be non-specific, and it implies collaboration between these two entities. Thus, events mediated by PML bodies may require PAN bodies as well.

Surprisingly, PAN bodies are recognized by an antibody raised against the FLAG epitope (see the Examples, below) (PAN bodies are seen in cells that do not express FLAG® protein). The FLAG® epitope is a synthetic epitope that consists of eight amino acid residues (DYKDDDDK (SEQ ID NO: 1)); Hopp et al. Biotechnology 6:1204-1210, 1988). The anti-FLAG® antibody is known in the art to react with many cellular proteins; but, in the context of the methods and materials described herein, recognition of the PAN bodies by the anti-FLAG® antibody has been extensively tested and is thus fortuitously illuminating these distinct, new nuclear bodies. It is possible that the anti-FLAG® antibody is reproducibly and specifically reacting with an epitope on an endogenous protein localizing within the PAN bodies, and this specific protein is induced by the exposure of cells to the MMLV (mouse murine leukemia virus) vectors as many of the cells tested have been. MMLV is mutated to be non-functional for use in a vector useful in transfecting cells with exogenous proteins. A recombination event could have resulted in the production of non-crippled virus, and this was explored by adding media conditioned by incubation of FLAG® positive cells (PAN body-containing cells) to cells in which the PAN bodies are not evident. After four days, incubation with conditioned media successfully induced PAN bodies in these cells suggesting that the PAN body inducing agent is a transferable agent (e.g., a virus or microorganism; see also, the Examples). The cells remain healthy and continue to grow well in culture. Thus, the presence of PAN bodies indicates or marks infected cells. Given this evidence supporting PAN bodies as an indicator of infection, identification and monitoring of PAN body formation can be useful in diagnosing cells for an infection. This use is described further herein, and it is consistent with the observation that PML domains are required for Herpes Simplex Virus Type 1 (HSV-1) replication (Lukonis et al., J. Virol. 71:2390-2399, 1997).

The Relationship of PAN Bodies to Other Nuclear Bodies

As their number and appearance are reminiscent of certain known intra-nuclear structures, the relationship of the PAN bodies to SC35 domains (prominent snRNP speckles) and Cajal bodies was also examined. SC35 domains are large, irregular domain of proteins located within the nucleus that can vary in size from 0.5 to 3.0 microns in diameter (see Moen et al., Hum. Mol. Genet. 4:1779-1789, 1995). Functionally, SC35 domains are enriched with a host of factors involved in pre-mRNA production. Cajal bodies are also nuclear structures, and they are implicated in a number of cellular processes, including the metabolism of both pre-mRNAs and nucleolar RNAs. More specifically, they are implicated in the maturation of splicing small nuclear ribonucleoproteins and the assembly of transcription complexes (Gall, Science 254:1371-1374, 2000). Components of the Cajal bodies include p80 coilin, splicing snRNAs, and many small nuclear ribonucleoprotein (snRNP)-specific proteins. Cajal bodies have been found to physically move within the nucleus and thus may be involved in mediating some forms of transport or directed movements of snRNPs in different parts of the nucleus. Cajal bodies also contain proteins involved in other functions, such as nucleolar functions, tumorigenesis, and cell cycle regulation. More generally, Cajal bodies may serve as centers for the assembly of multiple classes of macromolecular complexes.

A number of the methods described herein require localization of PAN bodies and rely on an assessment of the number, size, and proximity of those bodies to other nuclear structures. Any of the proteins specifically associated with PML bodies, SC35 bodies, or Cajal bodies, including those described in the paragraphs above, can be used to identify PML bodies, SC35 bodies, or Cajal bodies and thereby provide a visual point of reference for assessing PAN bodies.

As described further in the Examples below, PAN bodies do not overlap with SC35 or PML bodies, suggesting that they are not a member of either body and thus represent a separate nuclear domain that instead associates with SC35 and PML bodies. Importantly, however, there is a specific relationship between the PAN bodies and the other established nuclear domains (and this relationship can be examined to determine whether a cell is healthy or diseased or whether a potential therapeutic agent has a beneficial effect on an infected or malignant cell).

Scoring a relationship in cells that have both well-formed PAN bodies and SC35 domains show that greater than 80% of PAN bodies are adjacent to a prominent SC35 domain. The relationship of PAN bodies with PML bodies is particularly striking since PML bodies are smaller than SC35 domains and occupy less nuclear space. PAN bodies, which have a similar size and shape to the round PML bodies, are typically paired in a 1:1 stoichiometry with the PML bodies (FIG. 2C). In several experiments scored, the frequency of PAN bodies that associated with a PML body (as seen with anti-Daxx staining) is between ˜67-76%. A similar fraction (up to 76%) of PML bodies was shown to be associated with PAN bodies. Although PAN bodies and PML bodies are clearly adjacent and appear in close contact with one another, they are not coincident and thus they are distinct structures (or distinct parts of a compound structure).

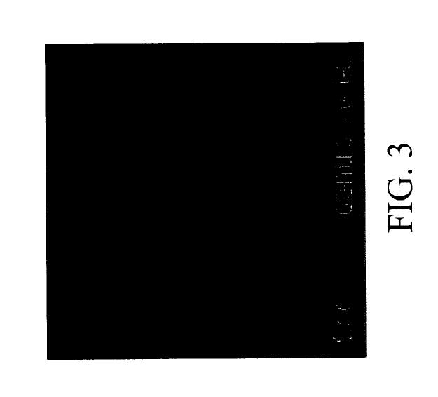

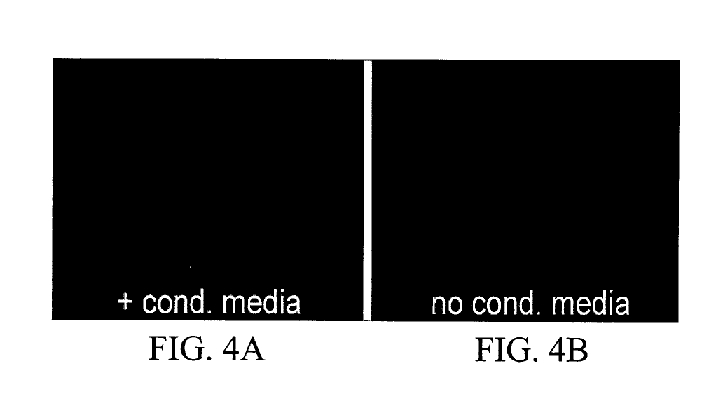

In contrast, staining for telomeres, which have a similar size and number to PML bodies, show little to no juxtaposition to PAN bodies. Since there is evidence that centromeres associate with PML bodies in some cells (Everett et al., J. Cell Sci. 112:3443-54, 1999), the relationship of centromeres relative to PAN bodies was of interest and was examined. Again, results show that there are only occasional instances of one or two centromeres adjacent to PAN bodies in some cells, but they are not seen to coincide (FIG. 3). The lack of any clear relationship of PAN bodies to telomeres or centromeres further suggests that the highly associated SC35 domains and PML bodies with PAN bodies reflects a specific association and not merely nuclear domains residing adjacent to one another by random chance. The non-random nature of this is also apparent in the 1:1 pairing typically seen of PAN and PML bodies.

Since SC35 domains and PML bodies both preferentially position adjacent to PAN bodies, triple-label analysis was done to visualize all three intra-nuclear structures simultaneously. PAN bodies, PML bodies and SC35 domains are commonly associated with one another in a 1:1:1 stoichiometry, most often forming a “triad” of closely opposed domains in a triangular arrangement. The SC35 domain only infrequently appears between the PAN bodies and PML bodies. While it is generally a coupled PAN body/PML body doublet that associates with the SC35 domain, it is variable whether it is the PAN body or the PML body that is closest to SC35. Optical sectioning, deconvolution and 3-D rendering of several cells confirmed the spatial relationship of these three structures to one other. This analysis also shows that PAN bodies collectively occupy less than 1% (e.g., 0.2, 0.4, 0.5, 0.5, or 0.8%) of the nuclear volume, with a similar estimate obtained for PML bodies. The very tiny volume occupied by individual bodies reinforces our conclusion that the pattern of juxtaposition of PAN bodies next to PML bodies does not occur by random chance.

The close coupling of PAN bodies and PML bodies evokes broader implications for the complicit involvement of these nuclear structures in regulating cell growth and proliferation, and in preventing or contributing to oncogenesis. The observations that PAN bodies appear similar in number, size and distribution to the PML bodies, and with high probability appears as a twin structure juxtaposed or conjoined to the PML body, strongly suggests that the two have some functional relationship to one another.

A number of different types of nuclear bodies were first identified by electron microscopy decades ago (Brasch and Ochs, Exp. Cell Res. 202:211-23, 1992), however the potential importance of these was not widely appreciated until the PML protein was found to localize in a class of spherical bodies (Ascoli and Maul, J. Cell Biol. 112:785-795, 1991). The PML protein is fused to the retinoic acid receptor in the t(15;17) translocation of acute promyelocytic leukemia (APL) (de The et al., Cell 66:675-84, 1991; Goddard et al., Science 254:1371-1374, 1991; Kakizuka et al., Cell 66:663-674, 1991; Pandolfi et al., Oncogene Oncogene 6:1285-1292, 1991) and the breakdown of PML domains is a major hallmark of APL (Dyck et al., Cell 76:333-343, 1994; Weis et al., Cell 76:345-356, 1994). To date over a dozen proteins have been found in PML bodies, including proteins involved in tumor suppression (pRB, PML, p53), growth control, apoptosis (p53), transcription regulation (p53, pRB, Daax, Sp100, Sp140), translation initiation (eIF-4, INT-6) (reviewed in Zhong et al., Nat. Cell Biol. 2:E85-90, 2000) and DNA replication and repair (BLM) (Bischof et al., J. Cell Biol. 153:367-80, 2001). Importantly, there is evidence that p53 localizes to PML bodies via interaction with PML and that this localization is necessary for p53 to act as a transcriptional co-activator of certain genes (Guo et al., Nat. Cell Biol. 2:730-6, 2000).

Since the discrete PAN bodies are not present in all cells of a given in vitro culture, it will be important to investigate whether concentration of PAN bodies at discrete sites relates to its cellular life-cycle and turnover within the cell. Recently, enzymes involved in protein modification and turnover (SUMO and proteasome activator PA28) have been linked to PML bodies (Fabunmi et al., J. Cell Sci. 114:29-36, 2001). Thus, complementary functions may occur in the paired PAN bodies and PML bodies that relate to protein modification and turnover for factors involved in cell signaling and regulation.

Based on the discovery that PAN bodies are spatially associated with PML bodies, which have been implicated in viral infection and cancer, we have developed methods of detecting infected or oncogenic cells (or cells that are not yet overtly cancerous, such as cells having some degree of dysplasia) by virtue of the presence of PAN bodies. Similarly, the appearance of PAN bodies within cells can provide the basis for assays that mark or monitor infected or oncogenic cells or identify infectious or carcinogenic agents. Assays that detect PAN bodies can also be used to screen agents (“agents” is a broad term encompassing virtually any type of composition, e.g., a chemical or biological agent having any degree of complexity (e.g., a protein, peptide, nucleic acid, or small molecule, or complexes or mixtures thereof)) for potential anti-viral or chemotherapeutic application. The agents may be screened for prophylactic application, therapeutic application, or both.

The identification and monitoring of PAN bodies in a cell can be used as a diagnostic for infection or cancer. As described above, the presence of PAN bodies in a cell can indicate infection (e.g., an infection with HSV, MMLV, or virally induced cancer). Use as a diagnostic would involve comparison of a normal cell with a test cell, which may be (or by independent indicators is known to be) infected or cancerous. PAN bodies can be detected by the methods described here (see the Examples). Once the cells are stained, the PAN bodies are then visualized by microscopy (e.g., light or fluorescent microscopy). Alternatively, PAN bodies can be visualized by electron microscopy. As a diagnostic or in monitoring cells, the presence of PAN bodies in the test cells but not in the normal control cells would indicate the presence of an infection or a cell that has lost control of its ability to proliferate or differentiate. The infection can be by any pathogen, including viruses such as HSV-1 or MMLV or a virus associated with cancer (e.g., a herpesvirus). Monitoring would require the collection of cells at different time points and analysis as described herein to determine the time course of infection for example. Numerous studies have shown a variety of viruses (including HPV, HSV, CMV, adenovirus) interact with the periphery of PML bodies and, in most but not all cases, disrupt them (Everett, 2001; Regad and Chelbi-Alix; 2001). Cells infected with (or suspected of being infected with) any of these virus types can be used in the methods described herein.

The detection of PAN bodies can be a method of screening for potential therapeutic agents. Using the methods infra to identify the presence of PAN bodies in cells, a test compound can be applied to a cell in which PAN bodies are present and the disappearance or disruption of the PAN bodies can indicate that the test compound is a potential therapeutic agent.

The candidate compound can be essentially any type of chemical or biological entity, and those of ordinary skill in the art will be able to identify sources of compounds to be tested in the methods described herein. There have been recent advances in high throughput screening, and those advances have given rise to a need for large numbers of compounds. Those of ordinary skill in the art routinely acquire and screen thousands of compounds in search of useful therapeutic agents. Compound libraries can be generated or obtained from a commercial supplier. For example, LeadQuest®, a library containing more than 80,000 compounds, can be obtained from Tripos (St. Louis, Mo.). Standard or custom made libraries can also be obtained from, for example, Ab Initio PharmaSciences (Basel, Switzerland), Affymax Research Institute (Santa Clara, Calif.), Array BioPharma, Inc. (Boulder, Colo.), Ascot Fine Chemical (Cambridge, England), ASDI Biosciences (Newark, Del.), BioLeads GmbH (Heidelberg, Germany), and BIOMOL Research Laboratories, Inc. (Plymouth Meeting, Pa.). The compounds may be chiral compounds, small heterocycle motifs, peptidomimetics, or natural product derivatives.

When in the form of a library, the library can be a biological library (of, for example, peptides, oligonucleotides, or antibodies), or a spatially addressable parallel solid phase or solution phase Examples of methods for the synthesis of molecular libraries can be found in the art, for example in: DeWitt et al. (Proc. Natl. Acad. Sci. USA 90:6909,1993); Erb et al. (Proc. Natl. Acad. Sci. USA 91:11422, 1994); Zuckermann et al. (J. Med. Chem. 37:2678, 1994); Cho et al. (Science 261:1303, 1993); Carrell et al. (Angew. Chem. Int. Ed. Engl. 33:2059, 1994); Carell et al. (Angew. Chem. Int. Ed. Engl. 33:2061, 1994); and Gallop et al. (J. Med. Chem. 37:1233, 1994).

Libraries of compounds may be presented in solution (e.g., Houghten, Bio/Techniques 13:412-421, 1992), or on beads (Lam, Nature 354:82-84, 1992), chips (Fodor, Nature 364:555-556, 1993), bacteria (U.S. Pat. No. 5,223,409), spores (U.S. Pat. Nos. 5,571,698; 5,403,484; and 5,223,409), plasmids (Cull et al. Proc. Natl. Acad. Sci. USA 89:1865-1869, 1992) or on phage (Scott and Smith, Science 249:386-390, 1990; Devlin, Science 249:404-406, 1990; Cwirla et al., Proc. Natl. Acad. Sci. USA 87:6378-6382, 1990; and Felici, J. Mol. Biol. 222:301-310, 1991).

Where inhibitors of gene expression are assayed, the inhibitor can be an antisense oligonucleotide or an RNAi. RNAi (RNA interference) refers to the process of introducing a homologous double stranded RNA (dsRNA) into a cell to specifically target a gene sequence, resulting in null or hypomorphic phenotypes. RNAi is interesting because it is a double stranded molecule, rather than single-stranded antisense RNA; it is highly specific; it is remarkably potent (only a few dsRNA molecules per cell are required for effective interference); and the interfering activity (and presumably the dsRNA) can cause interference in cells and tissues far removed from the site of introduction. Antisense oligonucleotides can also be tested as antiviral agents according to the methods of the invention and are well known in the art. Nucleic acids that hybridize to a sense strand (i.e., a nucleic acid sequence that encodes protein, e.g., the coding strand of a double-stranded cDNA molecule) or to an mRNA sequence are referred to as antisense oligonucleotides. While antisense oligonucleotides are “antisense” to the coding strand, they need not bind to a coding sequence; they can also bind to a noncoding region (e.g., the 5′ or 3′ untranslated region). For example, the antisense oligonucleotide can be complementary to the region surrounding the translation start site of an mRNA (e.g., between the −10 and +10 regions of a target gene of interest or in or around the polyadenylation signal). Moreover, gene expression can be inhibited by targeting nucleotide sequences complementary to regulatory regions (e.g., promoters and/or enhancers) to form triple helical structures that prevent transcription of the gene in target cells (see generally, Helene, Anticancer Drug Des. 6:569-84, 1991; Helene, Ann. N.Y. Acad. Sci. 660:27-36, 1992; and Maher, Bioassays 14:807-15, 1992). The sequences that can be targeted successfully in this manner can be increased by creating a so-called “switchback” nucleic acid. Switchback molecules are synthesized in an alternating 5′-3′, 3′-5′ manner, such that they base pair with first one strand of a duplex and then the other, eliminating the necessity for a sizeable stretch of either purines or pyrimidines on one strand of a duplex. Fragments having as few as 9-10 nucleotides (e.g., 12-14, 15-17, 18-20, 21-23, or 24-27 nucleotides) can be useful in the screening methods described herein.

Method of Identifying a Marker or Mechanism of Disease

The detection of PAN bodies can also be used to identify a marker of disease and once a marker of disease is identified, then the study of potential disease mechanisms is possible. Again using the methods described, infra, diseased cells can be compared to normal control cells by whether or not PAN bodies are detectable. Some diseases may not show this difference, however, for those that do show PAN bodies only in the diseased cells, the detection of the PAN bodies can thus serve as a marker for that disease. A marker of disease can be monitored throughout the course of a disease as a means of studying the mechanism of that disease. PAN body detection is thus useful in identifying a marker or mechanism of disease.

Method of Monitoring Disease

Once it has been established that PAN bodies are present in diseased cells, e.g., infected by a virus or micro-organism, or cancerous, obtaining cells from the diseased subject over the course of the disease and using the methods supra can be a means of monitoring a disease. For example, it is possible that the disappearance or disruption of the PAN bodies signifies remittance from the disease.

### EXAMPLES

### Example 1

We began this study with an intention to localize the c-Myc protein using several different epitope-tags (FLAG, HA, and GFP). Tagged Myc genes were introduced into null rat fibroblast cells using an MMLV derived viral vector that is incapable of replicating itself and requires a BOSC23 packaging cell line to produce virus capable of infecting cells. An anti-FLAG antibody delineated very bright and discrete nuclear foci in most cells (up to about 90%). In numerous side-by-side comparisons, neither the rat fibroblast parental line nor the myc null fibroblasts showed this staining. Upon finding anti-FLAG bound bodies (which we now know are the PAN bodies described herein but were termed Myc bodies in our earlier filed provisional application), we used immunofluorescence to determine whether the PAN bodies were coincident with SC35 domains, PML bodies, or Cajal Bodies. We found that all of these structures were clearly different and distinct entities.

We have since examined Myc distribution using three other methods and these results, and others, indicate that the bright foci seen with the anti-Flag antibody does not represent Flag-Myc. Nevertheless, the anti-Flag staining is fortuitously illuminating an important and unexplored nuclear structure: the PAN body. The Myc-bodies (again, these are now referred to as PAN bodies) do not appear following staining with either HA or GFP tags or anti-Myc antibodies. We began to understand better the nature of the PAN antibodies when we used the anti-Flag antibody on rat fibroblast cells carrying the HA-tagged or GFP-tagged constructs. Surprisingly, these cells contained unambiguous bright bodies detected with the Flag-ab even though they contained no flag-tagged protein (as confirmed by Western blots). In contrast, the TGR rat fibroblast parental line and the myc null cells, the two parental lines, never showed this Flag-staining in dozens of experiments. These results support the conclusion that the monoclonal anti-Flag antibody detects an endogenous protein that localizes to discrete nuclear bodies.

In addition, we induced PAN bodies by exposing cells to MMLV viral vectors. We stained three additional cell lines into which cyclins had been introduced using MMLV vectors. Two of the three cell lines contain the FLAG foci, and we postulate that this correlates with use of a different packaging cell line and possibly some recombination event that resulted in production of non-crippled virus.

The staining pattern is most prevalent in more confluent cultures (up to 95% of nuclei), suggesting that the appearance of these foci could be due to the spread of some signal, like a protein, or external trigger, such as a virus, within the cell cultures. We explored this possibility by adding media conditioned by incubation with flag foci positive cells to the parental rat fibroblasts and the myc null cells. Remarkably, the exposure of parental cells (which had been consistently negative) to the conditioned media for 4 days resulted in strong positive staining for flag foci.

While these results strongly suggest an infectious agent, we emphasize two points. First, all of these cell lines are healthy and grow well in culture. They also recently tested negative for mycoplasma. Therefore, we are not examining a phenomenon in “sick” cells. Second, although we see the bodies using an anti-Flag antibody only in some cultures, it is very possible if not likely that the body actually exists in other cells but the detected antigen is only seen in cells possibly exposed to the virus or packaging cell line. The studies were carried out as follows.

Cell lines and growth conditions. All cell lines are derivatives of the Rat-1 cell line (Prouty et al., Oncogene 8:899-907, 1993) and were propagated as described by Mateyak et al. (Mol. Cell Biol. 19:4672-4683, 1999). For microscopic observation, cells were grown on glass cover slips. A 10-amino acid flag epitope (MDYKDDDDKP (SEQ ID NO: 1)) was added directly in front of the initiator methionine residue of murine c-Myc using PCR and the tagged cDNA was cloned into the pLXSH retrovirus vector (Miller et al., Methods Enzymol. 217:581-599, 1993). Virions were prepared by transfection of the BOSC23 packaging cell line (Pear et al., Proc. Natl. Acad. Sci. USA 90:8392-8396, 1993) and used to infect HO15.19 (c-myc −/−) cells as described by Mateyak et al. (Mol. Cell Biol. 19:4672-4683, 1999. Following hygromycin selection, individual drug-resistant colonies were cloned with cloning rings and expanded in the absence of drug.

Cell fixation. Coverslips were rinsed twice with Hanks balanced salt solution (HBSS) followed by ice-cold cytoskeletal buffer (CSK: 10 mM PIPES pH 6.8, 100 mM NaCl, 300 mM sucrose, 3 mM MgCl₂,). Cells were extracted for 3-5 minutes in ice-cold CSK containing 0.5% Triton X-100 (Roche, Indianapolis, Ind.) and 20 mM vanadyl ribonucleoside complex (Life Technologies, Rockville, Md.), and subsequently fixed in 4% paraformaldehyde in Dulbecco's phosphate-buffered saline (PBS, pH 7.4) for 10 minutes at room temperature. Coverslips were stored in 70% ethanol at 4° C. Variations of this protocol as well as other protocols were tried, including fixation in paraformaldehyde followed by Triton permeabilization in CSK buffer, permeabilization by freeze-thawing (Kurz, 1986), or fixation in 100% cold methanol. We also tested whether storage of coverslips in PBS or 70% ethanol impacted the staining. In all cases, staining was compared to negative control cells treated identically. The protocol cited above gave significantly clearer specific staining than other protocols tried.

Immunostaining. The following antibodies were used: anti-flag M2 monoclonal antibody (Sigma Chemical Co., St. Louis, Mo., Cat. # F3165, 8 μg/ml), goat anti-Daxx (Santa Cruz Biotechnology, Santa Cruz, Calif., Cat. # sc-7000, 1 μg/ml), rabbit anti-SRm300 (Blencowe et al., 2000), rabbit anti-PML, rabbit anti-c-Myc (Upstate Biotechnology, Lake Placid, N.Y. Cat. # 06-340, 10 μg/ml), human anti-centromere autoimmune serum GS (Earnshaw and Rothfield, Chromosoma 91:313-321, 1985). Cells were blocked with 1% bovine serum albumin in PBS (PBS-BSA), incubated with antibodies (diluted in PBS-BSA) for 1 hour at 37° C., and washed successively (10 min each) in PBS, PBS with 0.1% Triton X-100, and PBS on a shaker. Fluorochrome-conjugated secondary antibodies were diluted 1:500 in PBS-BSA and incubated with cells for 1 h at 37° C. Finally, coverslips were washed as indicated above and mounted with Vectashield (Vector Laboratories, Burlingame, Calif.).

Telomere hybridization. Telomeres were visualized as described by Cornforth et al., Radiat. Res. 120:205-212, 1989). Antibody staining of protein epitopes was done before the DNA hybridization. After the secondary antibody treatment, the cells were re-fixed for 10 minutes in 4% paraformaldehyde and rinsed in PBS. Cells were heat denatured for DNA hybridization using a biotinylated telomere oligonucleotide probe (TTAGGT (SEQ ID NO: 2)) as described previously (Johnson et al., Methods Cell Biol. 35:73-99, 1991).

Microscopic Analysis. Microscopy was carried out with a Zeiss (Thornwood, N.Y.) Axioplan microscope with 100× plan-apo 1.4 objectives, triple band-bass filter sets (63000, Chroma, Brattleboro, Vt.) and a z-axis motorized stage (LEP, Hawthorne, N.Y.). Digital images were cquired by either a Photometrics (Tuscon, Ariz.) series 300 CCD or a Photometrics Photometrics Quantix camera. Image acquisition and analysis were done using Metamorph imaging software (Universal Imaging Corp., West Chester, Pa.).

Characterization of flag-Myc cell lines. In clonal lines expressing a flag-tagged c-Myc transgene inserted into c-myc −/− fibroblasts, expression of the flag-Myc transgene completely reversed the characteristic enlarged morphology of c-myc −/− cells. We showed that the introduction of the flag-Myc transgene does not perturb cell size by measuring forward scatter in a flow cytometric analysis of cells in suspension. An important phenotype of myc −/− cells is a strong impairment of proliferation, which is accompanied by a significant reduction in S phase content (Mayeyak et al., Cell Growth Differ. 8:1039-1048, 1997). Proliferation rate measurements of exponentially growing cultures showed that the flag-Myc transgene effectively restored the slow growth phenotype of c-myc −/− cells. Furthermore, flow cytometric analysis showed that flag-Myc completely restored the cell cycle DNA content profile to that of parental c-myc +/+ cells.

To determine protein expression levels (the protein bound by the anti-FLAG antibody), extracts of parental c-myc +/+ cells, c-myc −/− cells, and c-myc −/− cells expressing flag-Myc were immunoprecipitated with a monoclonal anti-Myc antibody (C-33) and subsequently immunoblotted with a polyclonal anti-Myc antibody (United Biomedical Inc., Hauppauge, N.Y.). This procedure is significantly cleaner and more sensitive than direct immunoblotting and was developed to effectively visualize low levels of Myc protein found in normal cells. The results clearly showed that flag-Myc was expressed at levels equivalent to that of endogenous c-Myc in parental Rat-1 c-myc +/+ cells. The flag-Myc protein displayed a slightly reduced electrophoretic mobility due to the presence of the flag epitope tag.

This analysis was performed on four clonal cell lines recovered from the flag-Myc infection of c-myc −/− cells with very similar results. The data were compiled from one cell line (clone 2) and are representative of all four cell lines. Clone 2 was chosen for further analysis because the expression level of flag-Myc was closest to that of endogenous Myc in Rat-1 cells; one clone showed a reduced level while two other clones had somewhat elevated levels.

In situ localization of PAN. The intracellular distribution of the protein recognized by the anti-FLAG antibody was investigated, relying on the highly antigenic flag-epitope to make the protein (initially believed to be c-Myc) more detectable by immunofluorescence. Several different fixation techniques were tested and the specificity of staining judged by comparison to two negative controls (RAT-1 c-myc +/+ cells and myc −/− parental cells). Clearly the best specific staining, seen in the flag-tagged cells but not the controls, was obtained using our standard procedure involving brief permeabilization with Triton followed by paraformaldehyde fixation and storage in 70% ethanol. Detection of the protein revealed a striking pattern of typically 10-20 (average=17) very bright foci that generally appeared as discretely bordered “domains”, 0.5-1 micron in diameter. These bodies were bright and easily visualized through the microscope without computer imaging. A spotted pattern of nuclear staining was seen in numerous experiments in many or most cells within a culture.

The number and size of the PAN bodies in flag-Myc cells varied between cells, with two distinct patterns apparent: nuclei with smaller, more numerous and dispersed spots, and nuclei with larger, more prominent domains that were fewer in number, typically between 10 and 20. Within the latter cells, the Myc domains displayed a regular morphology and appeared evenly distributed in the non-nucleolar nucleoplasm.

### Example 2

Subcellular fractionation has been an indispensable tool in the isolation of cytoplasmic organelles in order to study their function, structure, and biochemical properties. This process has recently been adopted for the isolation of intranuclear structures which has proven difficult since intranuclear structures are not enveloped by membranes and they can vary in their densities. There has been recent progress in the isolation of some intranuclear structures. Purification of human nucleoli and analysis by mass spectrometry has been reported recently (Andersen et al., (2002) Current Biology 12:1-11). Mass spectrometry analysis of nuclear fractions enriched for interchromatin granule clusters from mouse liver cells has also been reported (Mintz et al., (1999)EMBO J 18:4308-4320). Cajal bodies have also been reported (Wah et al., (2002) Molec. Biol. of the Cell 13:2461-2473). Based on the isolation of Cajal bodies a strategy for the isolation of PAN bodies is described herein in this example.

Buffers and Solutions. The magnesium concentrations and pH of the different buffer solutions can be varied to further optimize the isolation of PAN bodies. It has been reported that low magnesium concentration and a pH increasing from 7.4 to 8.5 allowed the isolation of another nuclear body, the Cajal body (Wah et al., (2002) Molec. Biol. of the Cell 13:2461-2473). This method relied on the strategy that separation from other nuclear bodies involves separation based on density (e.g., by Percoll/sucrose gradient by ultracentrifugation) and sensitivity to divalent ion concentration (e.g., Mg²⁺). Subsequently, varying the magnesium concentration and pH of the buffers described herein can allow the isolation of PAN bodies. Wah et al. (2002) reported that the fraction in which the Cajal bodies are identified includes very few if any PML bodies and other nuclear bodies. Thus, this procedure can be made suitable for the isolation of PAN bodies from Cajal bodies, PML bodies and other nuclear bodies, as the PAN bodies can be found in a different fraction.

All solutions contain EDTA-free complete protease inhibitor cocktail tablet (Roche Diagnostics, Mannheim, Germany), at a concentration of one tablet/50 ml. The compositions of the solutions can be as follows: S1 solution, 0.25 M sucrose, 10 mM MgCl₂; S2 solution, 0.35 M sucrose, 0.5 mM MgCl₂; S3 solution, 0.5 M sucrose, 25 mM Tris-HCl, pH 8.5; SP1 buffer, 1 M sucrose, 34.2% Percoll (Sigma, St. Louis, Mo.), 22.2 mM Tris-HCl, pH 7.4, 1.11 mM MgCl₂; SP2 buffer, 20% Percoll, 10 mM Tris-HCl, pH 7.4, 1% Triton X100 (BDH, Poole, England), 0.5 mg/ml heparin (Sigma); and HT buffer, 10 mM Tris-HCl, pH 7.4, 1% Triton X100, 0.5 mg/ml heparin.

Antibodies. The following antibodies can be used: anti-flag M2 monoclonal antibody (Sigma, St. Louis, Mo., Cat. # F3165, 8 μg/ml) to recognize PAN bodies, goat anti-Daxx (Santa Cruz Biotechnology, Santa Cruz, Calif., Cat. # sc-7000, 1 μg/ml) to recognize PML bodies, rabbit anti-SRm300 (Blencowe et al., 2000) to recognize SC35 domains, rabbit anti-PML (laboratory of John Sedivy) and human anti-centromere autoimmune serum GS (Earnshaw and Rothfield, 1985). Cells can be blocked with 1% bovine serum albumin in PBS (PBS-BSA), incubated with antibodies (diluted in PBS-BSA) for one hour at 37° C., and washed successively (10 minutes each) in PBS, PBS with 0.1% Triton X-100, and PBS on a shaker. Fluorochrome-conjugated secondary antibodies can be diluted 1:500 in PBS-BSA and incubated with cells for one hour at 37° C. Finally, coverslips can be washed twice with Hanks balanced salt solution (HBSS) followed by ice-cold cytoskeletal buffer (CSK: 10 mM PIPES pH 6.8, 100 mM NaCl, 300 mM sucrose, 3 mM MgCl2,) and mounted with Vectashield (Vector Laboratories, Burlingame, Calif.).

Sonication of HeLa Nuclei. All procedures described below can be performed at 4° C. unless stated otherwise. HeLa nuclei can be purchased from Computer Cell Culture Center (Seneffe, Belgium). After thawing, HeLa nuclei can be washed once with S1 solution (1400×g, 5 min). The nuclei can be then resuspended with S1 solution (8×10⁷ nuclei/ml) and overlaid on the same volume of S2 solution. After centrifugation (1400×g, 5 min) the pellet can be resuspended at 8×10⁷ nuclei/ml in 0.35 M sucrose 0.5 mM MgCl₂. The nuclei can be then sonicated with a Misonix 2020 sonicator fitted with a microtip and set at power setting 5. The energy can be given in 3 times 6-s pulses, with 6-s intervals between them. To ensure a reproducible delivery of energy, the sonicator is tuned according to manufacturer's instructions, and the nuclei can be consistently sonicated in 3-ml aliquots contained in a 15-ml Coming tube.

Enrichment of PAN bodies. For the following procedure, refer to the schematic below. After sonication, 0.42× volume of 2.55 M sucrose can be added to 1 volume of the sonicated nuclei, so that the resulting sucrose concentration is 1M. The nucleoli can be pelleted by centrifugation at 3000×g for 5 min in a GS-6 centrifuge (Beckman, Fullerton, Calif.), and washed once with S2 solution (1400×g, 2 min.). The supernatant, corresponding to the “nucleoplasmic fraction,” is carefully removed. One volume of the supernatant is mixed thoroughly with 0.8× volume of SP1 buffer. The final volume can be measured, and 20% (vol/vol) Triton X100 can be added, so that the resulting Triton X100 concentration is 1% (vol/vol). The mixture can be loaded into precooled SW41 tubes (Beckman, Palo Alto, Calif.) and centrifuged in a SW41 rotor (Beckman) at 37,000 rpm for 2 h. After ultracentrifugation, the tubes can be carefully unloaded from the top; the bottom 1 ml, containing a loose pellet, can be collected and designated as “P1” and the rest of the content can be designated “S1”. Pellet fractions can be pooled and mixed with 0.05× volume of 10 mg/ml heparin (Sigma Chemicals) and 600 U/ml DNasel (Sigma Chemicals). The sample can be incubated at room temperature for 30 min, and then mixed with 1× volume of SP2 buffer. The mixture can be loaded to precooled SW55 tubes and centrifuged in an SW55 rotor at 45,000 rpm for 1 hour. Apart from a loose pellet, a faint white band ˜2 cm above the bottom is also visible. The part of the gradient from the top to just above the white band can be carefully collected and designated as fraction “2A,” the white band is collected as fraction “2B,” and the rest of the material, including the pellet, is collected as fraction “2C.” Fractions 2B can be pooled and diluted 10 times with HT buffer. The diluted sample is divided into 1.5-ml aliquots and centrifuged at 14,000 rpm in a bench-top microfuge (Eppendorf) for 20 min. The pellets of all aliquots can be pooled and re-centrifuged so that all material from fraction 2B results in one pellet, which can then be resuspended in 0.5 ml of S3 solution. The resuspended pellet is centrifuged at 8000 rpm for 5 min in a bench-top microfuge (Eppendorf). The supernatant is carefully removed and designated as fraction 3S, and the pellet is fraction 3P. Fraction 3S, which has been reported to contain enriched Cajal bodies, is diluted 10 times with 25 mM Tris-HCl, pH 8.5, and is pelleted in a microfuge as above. Fraction 3P and any of the other fractions can be resuspended and diluted in Tris buffer to determine its contents by immunoblot and detection of PAN bodies with anti-FLAG antibody. Subsequent rounds of Percoll/sucrose gradient centrifugation and variation of pH and Mg²⁺ concentration can be performed to further enrich for PAN bodies.

Immunodetection. To detect the presence of PAN bodies, samples from each of the above fractions can be diluted 10 times in TM buffer (10 mM Tris-HCl, pH 7.4, 0.5 mM MgCl₂) and centrifuged in an Eppendorf (Hamburg, Germany) bench-top microfuge (14,000 rpm, 15 min). The pellets can be resuspended in <10 μl of TM buffer and spotted onto poly-L-lysine-coated glass microscope slides. The slides can be air-dried, rehydrated in PBS, and labeled with various antibodies according to a standard indirect immunofluorescence protocol (Lyon et al., (1997) Exp. Cell Res. 230:84-93). In some experiments, the preparations can be counterstained with Pyronin Y (Sigma Chemicals) after immunolabeling to reveal nucleoli.

For Western analysis, the pellets can be resuspended with Novex electrophoresis sample buffer (Invitrogen, Carlsbad, Calif.), separated in precast gradient polyacrylamide gels (Invitrogen), and blotted onto nitrocellulose membranes according to manufacturer's instructions. The membranes can be blocked in PBS containing 5% (wt/vol) skim milk (Marvel) and 0.1% Tween 20 (BDH) for 1 hour at room temperature and immunostained with various antibodies such as the anti-FLAG M2 monoclonal antibody (Sigma, St. Louis, Mo., Cat. # F3165, 8 μg/ml), goat anti-Daxx (Santa Cruz Biotechnology, Santa Cruz, Calif., Cat. # sc-7000, 1 μg/ml), rabbit anti-SRm300 (Blencowe et al., 2000), rabbit anti-PML. For Western blotting experiments, in which the amounts of protein loaded per lane can be standardized, the protein concentration of each sample is assayed by use of Coomassie Plus Protein Assay Reagent Kit (Pierce), according to manufacturer's instructions and using BSA as standard. The electrochemiluminescence signals can be detected with a CCD camera (Fujifilm LAS-1000; Fujifilm, Toyto, Japan) and quantified by use of Aida200 software (Raytest Isotopenmessgeräte GmbH, Straubenhardt, Germany).

To adapt the procedure suitably to isolate PAN bodies, samples from each fraction can be tested by Western blotting of each fraction using an anti-FLAG antibody followed by secondary antibody as describe, supra. Fractions containing higher levels of PAN bodies can be further enriched by repeating the above steps, varying the pH and Mg²⁺ levels, and continuing to detect the presence of the PAN bodies in each fraction.

Microscopy. Microscopic examination can be carried out using Zeiss (Thornwood, N.Y.) axioplan microscopes with 100× plan-apo 1.4 objectives, triple band-pass filter sets (63000, Chroma, Brattleboro, Vt.), and a Z-axis motorized stage (LEP, Hawthorne, N.Y.). Digital images can be acquired acquired by either a Photometrics (Tucson, Ariz.) Series 300 CCD or a Photometrics Quantix camera. Image acquisition and analysis can be done using Metamorph imaging software (Universal Imaging Corp., West Chester, Pa.).

For transmission EM (TEM) studies, HeLa cells can be pelleted in a microfuge and lightly fixed with 4% paraformaldehyde in PBS for 10 min before they are immunolabeled with anti-FLAG (5P10, undiluted hybridoma supernatant, or 204/10, 1:250) 10-nm gold-conjugated secondary antibodies (1:25). Blocking and antibody dilution buffer is PBS, 0.5% goat serum, 0.1% Tween 20, 1% BSA. Labeled cells can be embedded in standard epoxy resin (Durcupan, Sigma) embedding techniques. To analyze isolated PAN bodies, the fraction containing highly enriched PAN bodies can be loaded onto poly-L-lysine-coated glass coverslips; the PAN bodies can be labeled with anti-FLAG antibodies and detected using a combination of fluorescence and gold-conjugated secondary antibodies. Coverslips can be examined in the fluorescence microscope, and areas containing a high concentration of labeled PAN bodies can be located. Coverslips can be then fixed in 80 mM PIPES/KOH, pH 6.8, 1 mM MgCl₂, 1 mM EGTA, 150 mM sucrose, 0.25% glutaraldehyde, and 2% paraformaldehyde; washed in PBS and then in H₂O; postfixed in 1% osmium tetroxide in H₂O for 20 min at room temperature; washed in H₂O; dehydrated in 70% ethanol for 10 min; stained in 1% uranyl acetate in 70% ethanol for 20 min; washed 2 times in 70% ethanol; and further dehydrated through 90, 95, and 100% ethanol and propylene oxide before they are flat-embedded in epoxy resin (Durcupan). Coverslips can be removed of the resin by brief immersion in liquid nitrogen. The coverslips could then be snapped off the surface of the resin. Thin sections can be cut (Reichart-Jung Ultracut UCT, Leica Microsystem, Nussloch, Germany) and stained with lead citrate before they are examined with a Joel 1200Ex transmission electron microscope (Tokyo, Japan).

For field emission scanning EM (FESEM), samples can be prepared according to methods described by Goldberg and Allen (1992) J. Cell Biol. 119: 1429-1440. Briefly, purified PAN bodies can be resuspended in 10 mM Tris-HCl, pH 8.5, and loaded onto poly-L-lysine coated silicon chips (Agar Scientific Ltd, Stansted, United Kingdom). Unfixed PAN bodies can be labeled with anti-FLAG antibody and 15 nM gold-conjugated secondary antibodies before they are fixed using SEM fix (80 mM PIPES/KOH, pH 6.8, 1 mM MgCl₂, 1 mM EGTA, 150 mM sucrose, 0.25% glutaraldehyde, 2% paraformaldehyde). Labeled PAN bodies can then be dehydrated through a graded ethanol series (70, 90, 95, and 3 times 100%) and then into 100% acetone before they are critical-point dried (Bal-Tec CPD 030, Balzers, Switzerland). Dried specimens can be coated with 1.5 nM of chromium and examined in FESEM (Hitachi S4700, Tokyo, Japan).

### Example 3

Experimental Evidence of PAN Association with Nuclear Bodies

Using the anti-FLAG antibody to recognize PAN bodies and the anti-Daxx antibody to recognize PML bodies (described, e.g., below), photomicrographs were taken and the rate of association with PML bodies was scored. As mentioned, supra, the anti-FLAG antibody fortuitously recognizes the PAN bodies and anti-Daxx antibody recognizes a protein within the PML body, Daxx. An exemplary photomicrograph is shown in FIG. 2C. Approximately 40 cells were scored from photomicrographs. Of all the cells showing PML bodies, 82.5% showed PAN bodies associating with PML bodies. And, of the cells showing PAN bodies, 94% of them showed an association with PML bodies.

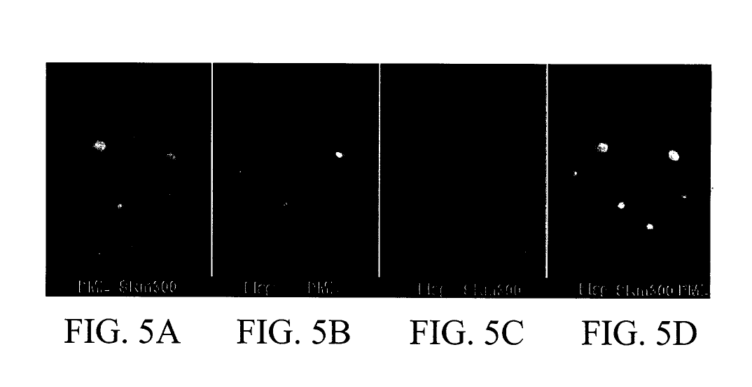

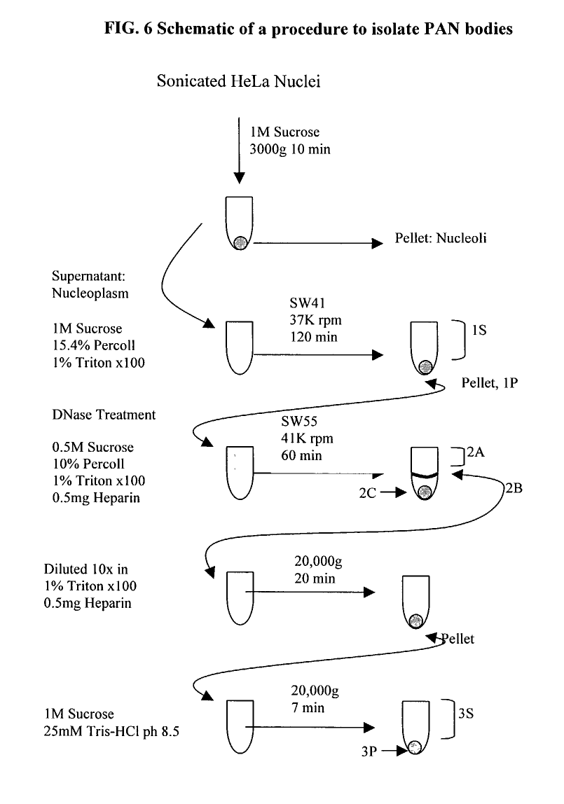

Of the total number of PAN body signals detected in a triple labeling of antibodies recognizing PAN bodies, PML bodies and SC35 domains (see FIG. 5A-5D), 97% of the PAN body signals scored were associated with PML body signals. The association here is specifically an adjacent association rather than colocalization within the PML bodies. These results indicate that PAN bodies are often found adjacent to PML bodies in the nucleus.

Triple labeling experiments also showed the rate of association of PAN bodies to both PML bodies and SC35 domains. PAN bodies were found associated simultaneously to both PML bodies and SC35 domains in one complex in 76% of signals detected, while association between PAN bodies and SC35 domains without PML domains represented 19% of signals and only 3% of signals detected represented PAN bodies associated with PML bodies alone, without SC35. Only 3% of signals represented PAN domains which were not associated with either PML bodies or SC35 domains. Conversely, 73% of the detectable PML body signals were found associated with both PAN bodies and SC35 domains in one complex while 20% of the detectable PML body signals were found associated with SC35 domains without PAN bodies. Only 3% of detectable PML body signals were associated with PAN bodies without SC35 domains while only 4% of detectable PML body signals were not associated with either PAN bodies or SC35 domains. When scoring for the SC35 domains which are much larger nuclear structures, SC35 domains were scored differently because signals often had to be counted more than once due to the association of more than one PML body or PAN body signal to a single SC35 domain. 56% of the time, SC35 domains were seen associated with both a PML body and a PAN body in one complex. 14% of the time, SC35 domains were seen with PAN bodies and not PML bodies, 15% of the time SC35 domains were seen with PML bodies and not PAN bodies, and 15% of the time SC35 domains were not seen with either PAN bodies or PML bodies. In summary, PAN bodies are often found associated adjacently to PML bodies in a complex with SC35 domains.

In triple labeling experiments using antibodies recognizing PAN bodies, PML bodies and Cajal bodies, 6% of the PAN body signals were associated with both PML bodies and Cajal bodies in the same complex. 63% of PAN body signals were associated with PML bodies without Cajal bodies associated. 2% of PAN body signals were associated with Cajal bodies but not PML bodies. Conversely, 50% of Cajal body signals were found associated in a complex with PAN bodies and PML bodies while 12% of the Cajal body signals were associated with PAN bodies and not PML bodies. 28.6% of Cajal body signals were associated with PML bodies and not PAN bodies while 9.5% of Cajal bodies were not associated with either PAN bodies or PML bodies. In summary, Cajal bodies don't seem to be associated with any one nuclear body a mojority of the time. Cajal bodies seem to spend some time associated with PAN bodies, some time with PML bodies, and some time with both in a complex.

A number of embodiments of the invention have been described. Nevertheless, it will be understood that various modifications may be made without departing from the spirit and scope of the invention. Accordingly, other embodiments are within the scope of the following claims.

### Example 4

A number of specific proteins have been shown to associate with PML bodies when expressed by transient transfection. We will obtain clones that encode these proteins and transfect them into PAN-positive cells to determine if they produce foci that overlap the PAN bodies or PML bodies. Proteins that do will reside within or otherwise be tightly associated with PAN or PML bodies and antibodies or other markers that specifically bind these proteins can then be used to identify (or localize or study) PAN bodies (e.g., their number, size, or distribution) and their relationship to PML bodies. The proteins will include:

GRIP1: GRIP1, also known as TIF2 (transcriptional intermediary factor 2), was shown to form discrete intranuclear foci reminiscent of PAN foci in a subset of transfected cells. These foci, which were also enriched in components of the 26S proteasome, often associated with PML bodies (Baumann et al., Mol. Endocrinol. 15:485-500, 2001).

SV40 large T antigen: The large T protein of SV40, when introduced to cells by transfection of the SV40 encoding gene, has been found to accumulate in foci associated with PML bodies (Carvalho, 1995; Jiang, 1996).

BRCA1: When overexpressed, the BRCA1 protein has also been shown to accumulate in large foci closely apposed to PML bodies (Maul, 1998). Since most proteins do not do this even when overexpressed, it may suggest accumulation in a structure, rather than just a protein aggregate.

PLZF Spots: A particularly promising protein candidate is encoded by the gene involved in a translocation that leads to APL, the PLZF protein. PLZF concentrates in 10-20 dot-like structures, but according to one report, these do not overlap PML domains but are instead adjacent to them (Ruthardt et al., Oncogene 16:1945-1953, 1998).

What is claimed:

1. A method of identifying a PML-body adjacent nuclear body (PAN body) in a cell, the method comprising
a) contacting a cellular nucleus with a first antibody that specifically binds a nuclear body and a second antibody that specifically binds a protein within a PML body; and
b) determining whether the first antibody binds a nuclear body that is adjacent to a PML body that is bound by the second antibody, wherein a nuclear body located adjacent to a PML body is a PAN body.

2. The method of claim 1, wherein the first antibody is an anti-FLAG antibody.

3. The method of claim 1, wherein the nuclear body determined to be a PAN body is further characterized by
a) contacting the cellular nucleus with a third antibody that specifically binds a protein within an SC35 domain; and
b) determining whether the SC35 domain bound by the third antibody is adjacent to the PAN body.

4. The method of claim 1, wherein the nuclear body determined to be a PAN body is further characterized by
a) contacting the cellular nucleus with a third antibody that specifically binds a Cajal body; and
b) determining whether the Cajal body bound by the third antibody is adjacent to the PAN body.

5. A method of inducing a PAN body in a cell, the method comprising exposing the cell to (a) media conditioned by prior exposure to PAN-positive cells or (b) a pathogen.

6. The method of claim 5, wherein the cell is one in which no PAN bodies are evident prior to exposure to the conditioned media or pathogen.

7. The method of claim 5, wherein the cell is one in which PAN bodies are evident prior to exposure to the conditioned media or pathogen.

8. The method of claim 5, wherein the cell is a cell in culture.

9. The method of claim 5, wherein the cell is a cell in vivo.

10. The method of claim 5, wherein the cell is a human cell.

11. The method of claim 5, further comprising a step in which induced PAN bodies are visualized by exposing the cells, following exposure to the conditioned media or pathogen, to an antibody that recognizes the PAN body.

12. The method of claim 5, wherein the pathogen is a virus or bacterium.

13. The method of claim 12, wherein the virus is a leukemia virus.

14. A method of determining whether a cell has been exposed to a pathogen, is malignant, or is at risk of becoming malignant, the method comprising determining whether the cell expresses PAN bodies, the presence of PAN bodies indicating that the cell has been exposed to a pathogen, is malignant, or is at risk of becoming malignant.

15. The method of claim 14, wherein determining whether the cell expresses PAN bodies is carried out by an immunoassay or by immunohistochemistry.

16. The method of claim 15, wherein the immunoassay or immunohistochemistry is carried out by exposing nuclear material from the cell to an anti-FLAG antibody.

17. The method of claim 14, wherein determining whether the cell expresses PAN bodies is carried out by electron microscopy.

18. The method of claim 14, wherein the cell is a cell in culture.

19. The method of claim 14, wherein the cell is a human cell.

20. The method of claim 14, wherein the pathogen is a virus or bacterium.

21. The method of claim 20, wherein the virus is a leukemia virus.

22. The method of claim 14, wherein the malignancy is associated with a leukemia.

23. A method of screening a test compound to identify a potential therapeutic agent, the method comprising
a) providing a cell having a visible PAN body or a plurality of PAN bodies;
b) contacting the cell with the test compound; and
c) evaluating the effect of the test compound on the PAN body or PAN bodies, wherein disappearance of the PAN body, a reduction in the number of PAN bodies, or a disassociation between one or more PAN bodies and one or more PML bodies indicates that the test compound is a potential therapeutic agent.

24. The method of claim 23, wherein the test compound is a protein or peptide; a nucleic acid; or a chemical entity.

25. The method of claim 23, wherein the therapeutic agent is an anti-viral or chemotherapeutic agent.

26. A method of determining whether the state of PAN bodies within a diseased cell can serve as a marker for disease, the method comprising
a) providing an apparently healthy cell and a cell that is diseased; and
b) determining whether and, optionally, where, PAN bodies are expressed within the healthy cell and within the diseased cell, wherein a difference in PAN body expression between the apparently healthy cell and the cell that is diseased indicates that the state of PAN bodies is a marker for disease.

27. The method of claim 26, wherein the cell that is diseased is infected with a virus or is malignant.

28. The method of claim 26, wherein the state of PAN bodies in the diseased cell is assessed before the cell has been exposed to a therapeutic agent and after the cell has been exposed to a therapeutic agent, a change in the expression of PAN bodies to a state more reminiscent of the PAN bodies in the healthy cell indicating that the therapeutic agent is having a beneficial effect on the diseased cell.

29. The method of claim 28, wherein the therapeutic agent is an anti-viral agent or a chemotherapeutic agent.

30. An isolated PAN body.

31. A method of isolating a PAN body, the method comprising
a) providing cell nuclei;
b) disrupting the nuclei in a solution comprising divalent ions;
c) passing the disrupted nuclei over a Percoll-sucrose gradient at a first pH and subsequently passing the resulting eluate over a Percoll-sucrose gradient at a second pH, the second pH being higher than the first, to obtain enriched fractions; and
d) evaluating the enriched fractions for the presence of PAN bodies.

32. The method of claim 31, wherein the concentration of the divalent ions is about 0.5-1.0 mM.

33. The method of claim 32, wherein the divalent ions are magnesium ions.

34. The method of claim 31, wherein the disrupted nuclei are passed over the gradient between two and five times.

35. The method of claim 31, wherein the pH at the first passage is about 7.4 and the pH at the last passage is about 8.5.

36. A PAN body isolated by the method of claim 31.

37. The isolated PAN body of claim 36, wherein the PAN body, PML body, and SC35 bodies are associated with one another in about a 1:1:1 stoichiometry when present in the nucleus of a biological cell.

38. The isolated PAN body of claim 31, wherein the PAN body and the PML body are associated with one another in about a 1:1 stoichiometry when present in the nucleus of a biological cell.
